(19) United States
(12) Patent Application Publication
(10) Pub. No.: US 2023/0159485 A1
(43) Pub. Date: May 25, 2023

(54) Methods for Preparing Cannabinoids and Related Instruments

(71) Applicant: 3BC, LLC, Topsfield, MA (US)

(72) Inventor: Gregory A. Sotzing, Storrs, CT (US)

(73) Assignee: 3BC, LLC, Topsfield, MA (US)

(21) Appl. No.: 17/918,060

(22) PCT Filed: Apr. 9, 2021
(86) PCT No.: PCT/US2021/026586

§ 371 Date: Oct. 10, 2022

Related U.S. Application Data
(60) Provisional application No. 63/007,999, filed on Apr. 10, 2020.

(51) Int. Cl. C07D 311/92; C07D 311/86

(52) U.S. Cl. CPC C07D 311/92; C07D 311/86

(57) ABSTRACT

Methods and instrumentation for converting cannabidiol (CBD) and CBD-like compounds to other naturally-occurring or synthetic cannabinoids, such as THC, CBN and/or CBC, which processes may be solvent-free, Generally, the conversion of CBD is carried out in the presence of a Lewis acid, an oxidant or both, which may be present in catalytic amounts. A reaction may be a two-phase reaction with the Lewis acid present on a support material in a column or similar chamber through which CBD passes and is converted to the cannabinoids. The reactions allow direction of relative yields of certain cannabinoid products by altering the identity of the acid reagent.

### BACKGROUND OF THE DISCLOSURE

The present disclosure relates to cannabinoids, and more particularly, to a process for preparing cannabinoids, both naturally occurring and synthetic, from cannabidiol (CBD), which processes may be solvent-free.

Synthetic methods exist whereby a CBD oil is converted to naturally occurring cannabinoids that are derived downstream in the pathway in the biological process, such as for example, Δ-8-tetrahydrocannabinol (THC) and Δ-9 THC, cannabinol (CBN), and cannabichromene (CBC). Additional synthetic methods exist for deriving synthetic cannabinoids, such as cannabinoids coming from the hydroxylation and alkoxylation (ethoxylation, for example) of the cyclohexene ring of THC to generate hydroxyl-THC or ethoxyl-THC.

For example, US Patent Application Publication No. 2004/0143126 discloses a method for preparation of Δ-8 THC and Δ-9 THC from CBD that requires use of solvents.

Further, methodology and procedures for driving stereospecific conversion of CBD to major product Δ-8 THC using PTSA have been published. The published procedure requires use of toluene solvent in a 1:1 weight ratio with CBD. Toluene is a toxic, carcinogenic, high boiling organic solvent.

Such process necessarily requires the later removal of solvent before the Δ-8 THC and/or Δ-9 THC can be used further, which can present many challenges. Indeed, solvent removal technology is often novel methodology in and of itself. In addition, solvent use adds cost (for solvent and by increasing heat required to drive the reaction), increases toxicity and can increase impurities of product. This is especially the case for reactions that require conversion of a molecule of one polarity to a molecule of a substantially different polarity, as polarity of a solvent plays a critical role in the stabilization of the transition state of the mechanism, and this will substantially impact the kinetics of the reaction.

It would thus be useful to have a process for preparing different cannabinoids from CBD without use of solvents and/or by new preferably single-step reaction conditions that produce quantitative or near quantitative yields of desired product cannabinoids. It would additionally be useful to have an instrument or other equipment that assists in performing a solvent-free process.

### BRIEF DESCRIPTION OF THE DRAWINGS

FIG. 1 is an exemplary mechanism showing solvent-free treatment of CBD with a sulfonic acid;

FIG. 2 shows an exemplary reaction of CBD with a Lewis acid according to the disclosed embodiments;

FIG. 3 shows an exemplary reaction of CBD with an oxidant according to the disclosed embodiments;

FIG. 4 shows an exemplary reaction of CBD with oxidant and acid or an oxidant that also behaves as an acid according to the disclosed embodiments;

FIG. 5 is a proton NMR of the product resulting from reaction of neat CBD with FeCl₃; and

FIG. 6 shows exemplary conversion of a variety of specific CBD compounds to different oxidation level products.

### SUMMARY

Disclosed herein are methods for forming cannabinoids from CBD starting material without the use of solvents. A variety of CBD starting materials can be utilized, including for example, pure crystalline CBD, CBD from a plant source, and CBD oil with CBD present in a liquid at room temperature.

CBD may be substantially pure or impure, such as an oil derived from extraction from a plant source that necessarily includes other cannabinoids.

In one embodiment, pure CBD is converted to cannabinoids in the presence of an acid that behaves as a catalyst, or the acid reagent can be used in non-catalytic amounts.

In another embodiment, CBD is converted to cannabinoids in the presence of an oxidant reagent and acid reagent.

In another embodiment, CBD is converted to cannabinoids in the presence of an oxidant reagent that also behaves as an acid.

In another embodiment, CBD is converted to cannabinoids in the presence of an oxidant reagent and absence of an acid.

In another embodiment, CBD is converted to cannabinoids in the presence of a clay reagent.

The process may include melting or volatilizing pure CBD by heat in the presence of the reagent. For example, melted CBD can be stirred in the presence of catalyst or volatilized CBD can be processed through a solid-state column containing the reagent.

In another embodiment, the CBD starting material exposed to the reagent under elevated temperature is in an impure form such as within the plant or biomass or it can be in an oil.

In another embodiment, an instrument is provided for use in a volatilization process that employs an oven within which naturally occurring, pure CBD or another form of CBD is heated. A solid-state catalyst column or comparable component is positioned downstream from the oven. The volatized CBD oil is directed from the oven through the column to initiate the reactions, thereby converting the CBD to a cannabinoid that is later in the biological process (such as, for example, Δ-8 THC, Δ-9 THC, CBN and/or CBC). Alternatively, the oven may contain CBD on a solid-state catalyst support making the conversion of CBD to a downstream cannabinoid such as Δ-8 THC, Δ-9 THC, CBN and/or CBC happen within the oven component without use of a solid-state catalyst column.

In another embodiment, an instrument is provided that employs a clay support material that is optionally treated with another reagent through which CBD starting material is directed to pass, thereby converting CBD to cannabinoids.

Another embodiment provides a method for varying yield ratios of different cannabinoids comprising altering the reagent and optionally the amount. For example, different acids yield different ratios of cannabinoid products. In a preferred embodiment, different sulfonic acids are used to direct ratios of different cannabinoid products.

In another embodiment, the respective reaction(s) can be performed in a reactive distillation process wherein a column is packed with a catalyst or catalyst is present within a distillation flask. Here, the CBD starting material reacts with the catalyst during the distillation process to form the final product. Reactive distillation processes are widely applicable to the reactions disclosed herein, including, without limitation, conversion of CBD starting material to major product CBN in the presence of selenium oxide (SeO₂).

The disclosure provides for bulk reaction of CBD in presence of a Lewis acid reagent, which may behave as a catalyst. The disclosure also provides for two-phase reaction of CBD wherein the Lewis acid reagent is on a support material in a column or similar chamber through which CBD passes and is converted to cannabinoids, such as THC, CBN and/or CBC. A virtually unlimited variety of Lewis acid reagents can be employed within the disclosed methods. Moreover, the relative yields of certain cannabinoid products can be directed by altering the identity of the acid reagent.

Further, the acid can be present in an aqueous phase with CBD or a CBD oil as the organic phase.

In another embodiment, CBD starting material is converted to CBND in the presence of an oxidant and in absence of acid in a solvent-free reaction. See FIG. 3.

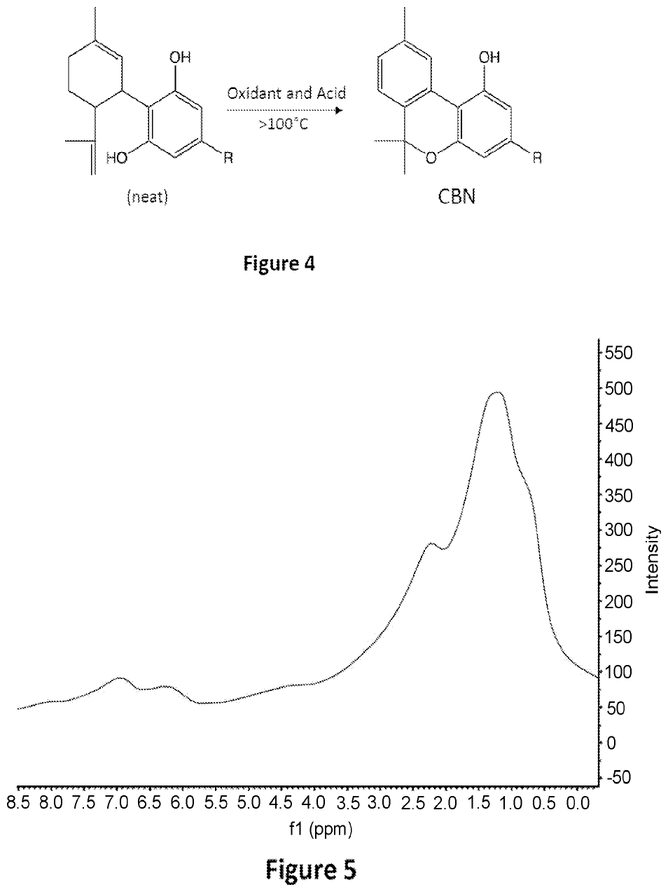

In yet another embodiment, CBD starting material is converted to CBN in the presence of an oxidant and acid or an oxidant that also behaves as an acid with optional use of a solvent. See FIG. 4.

In a preferred embodiment, CBD starting material is converted to CBN in the presence of an oxidant and acid or an oxidant that also behaves as an acid in a solvent-free reaction. See FIG. 4.

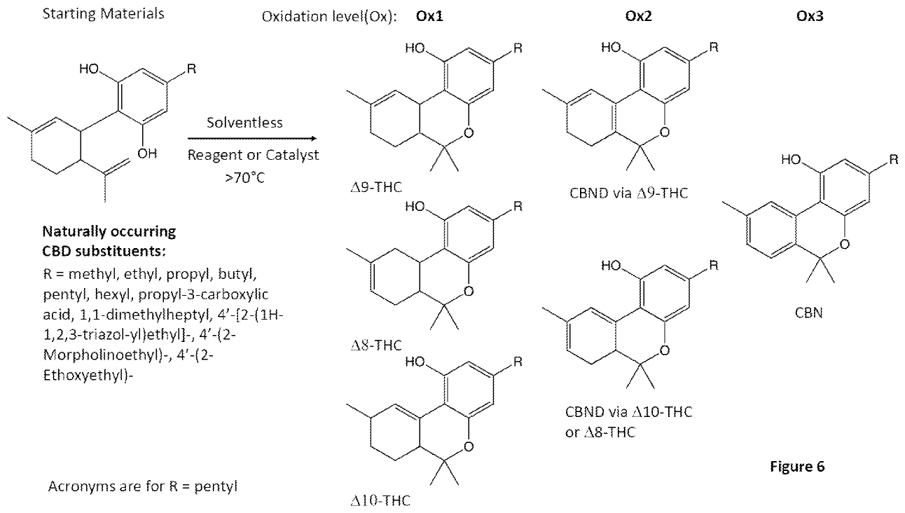

Notably, with reference to FIG. 6, the disclosed reactions and methodology are not limited to converting classic CBD (i.e., wherein R is pentyl). Alternative R groups include, for example, methyl, ethyl, propyl, butyl, pentyl, hexyl, propyl-3-carboxylic acid, 1,1-dimethylheptyl, 4′-[2-(1H-1,2,3-triazol-yl)ethyl]-, 4′-(2-Morpholinoethyl)-, and 4′-(2-Ethoxyethyl)-.

### DETAILED DESCRIPTION

Among the benefits and improvements disclosed herein, other objects and advantages of the disclosed embodiments will become apparent from the following. Detailed embodiments of a method of preparing cannabinoid compounds and related instrumentation are disclosed; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrase “in some embodiments” as used herein does not necessarily refer to the same embodiment(s), though it may. The phrases “in another embodiment” and “in some other embodiments” as used herein do not necessarily refer to a different embodiment, although it may. Thus, as described below, various embodiments may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described unless the context clearly dictates otherwise. In addition, throughout the specification, the meaning of “a,” “an,” and “the” include plural references. The meaning of “in” includes “in” and “on”.

Further, the terms “substantial,” “substantially,” “similar,” “similarly,” “analogous,” “analogously,” “approximate,” “approximately,” and any combination thereof mean that differences between compared features or characteristics is less than 25% of the respective values/magnitudes in which the compared features or characteristics are measured and/or defined.

As used herein, the term “significantly sterically hindered” acid means having a minimum t-butyl substituent, and would necessarily include larger substituents, such as, without limitation, adamantyl and larger polymer chains. In the disclosed embodiments, when insoluble, polymers such as PSSA and polyphosphonic acid, and clays, such as bentonite and kaolinite, make it such that the liquid CBD molecules have to approach and collide with the surface of the catalyst in the least sterically hindered manner.

Additionally, as used herein, the term “CBD” is intended to be given a broad interpretation covering CBD-like compounds with varying R groups (see FIG. 6), and not strictly limited specifically to cannabidiol (wherein R is pentyl). However, it is understood that cannabidiol may be and often is used. In cases wherein a CBD-like starting material with an alternate R group is employed, the respective reaction products would accordingly retain the alternate R group.

Alcohols substantially increase the polarity of a molecule, and a second alcohol functionality further enhances this polarity. The dielectric constant of the molecule in the liquid form scales with the polarity of the molecule. In the conversion of CBD to THC, there is a ring closure reaction of a phenolic hydroxy group converting it to an ether with the THC formation. In other words, for every conversion of CBD to THC, one alcohol functionality is lost. Upon 50% conversion, 75% of the alcohol functionalities remain in the reaction solution. Such a change causes a substantial decrease in polarity of the reaction solution, thereby leading to an instability in the transition state because formation of carbocation is the slow step of the mechanism, meaning that the carbocation would be best stabilized in polar solvents in accordance to the Hammond Postulate. Therefore, there is an even greater change in the polarity of such a reaction in the absence of solvent as compared to a reaction in solvent within which the reacting species can be less than 50% of the total weight of the reaction solution. At 100% conversion solvent-free conditions, there would be 50% alcohol functionalities remaining.

Understandably, the disclosed solvent-free methodology allows for significantly increased scale production of cannabinoids (i.e., kg or greater) at a substantially lower cost since solvent removal steps and advanced technology are omitted from the process. Conversion from pure CBD to a later cannabinoid, such as THC, also provides a means for a dry herb vaporizer to deliver different ratios of cannabinoids than that found in the plant by converting the CBD in the plant to THC. Such a device can be provided with an adjustable “volume” dial to vary the quantity of THC produced. Other cannabinoids, such as CBN and/or CBC, can be produced by varying the dry herb vaporization conditions.

The disclosed dry herb vaporizer operates by providing CBD on a solid support that is loaded into an oven chamber. Suitable solid supports for CBD include inorganic materials, such as silica or glass, or organic materials, such as polymers in the form of a covalent bond as would be for an ester or infused into a polymer like common plasticizers. Further, the CBD can be placed onto a solid-state Bronsted-Lowry acid reagent such as polystyrene sulfonic acid or a crosslinked version of this polymer that utilizes divinyl benzene. Alternatively, the CBD can be coated on a Lewis acid support such as Kaolinite or Montimorillonite clay. Application of heat volatizes the plasticizer with the volatilization delivery being controlled by the temperature and the variables of Fick's Law of Diffusion.

An alternative embodiment utilizes a naturally occurring herb rather than pure CBD. A plant such as hemp that comprises CBD, but not THC, could be vaped via the conversion of CBD to THC within the dry herb vaporizer. Alternative embodiments utilize an oil comprising CBD, but not substantially pure CBD.

As will be described in greater detail below, by altering conditions such as the reagent, reagent amount, surface area, flow rate, etc., the conversion process can be optimized. The temperature can be controlled in the oven to initiate volatilization and controlled separately in the solid-state column. The temperature in the column is controlled to control the amount of volatized CBD, and thus the conversion kinetics and efficiency to THC (or other cannabinoid(s)).

In addition to a small device for conversion of CBD to cannabinoids using either pure CBD on a support, CBD within an oil or CBD within a plant (hemp, marijuana, flaxseed), a larger scale device comprising a solid-state column containing reagent can be utilized for large scale conversions of CBD. This column can be positioned within the line of an extraction machine or it could be used just downstream from an oven that volatizes oil from a plant.

An exemplary two-phase reaction includes using polystyrenesulfonic acid coated on glass beads in the presence of CBD oil at elevated temperature to produce later cannabinoids. In this procedure, CBD oils can be volatized by heat out of the plant, or off from a solid support, or from the crystalline material. The volatized CBD passes through a solid-supported acid column such as polystyrenesulfonic acid coated on glass beads and the CBD is converted to Δ-8 THC, Δ-9 THC, CBN, and/or CBC depending on the temperature, time, flow rate, etc. being used. Additionally, as will be discussed further below, the relative yield of individual cannabinoid products can be altered by altering the acid reagent, and optionally the amount. In such a two-phase reaction, reactions can be carried out on impure systems, which allows for a boost in THC, CBN and/or CBC content from the CBD present in an oil mixture or in a plant.

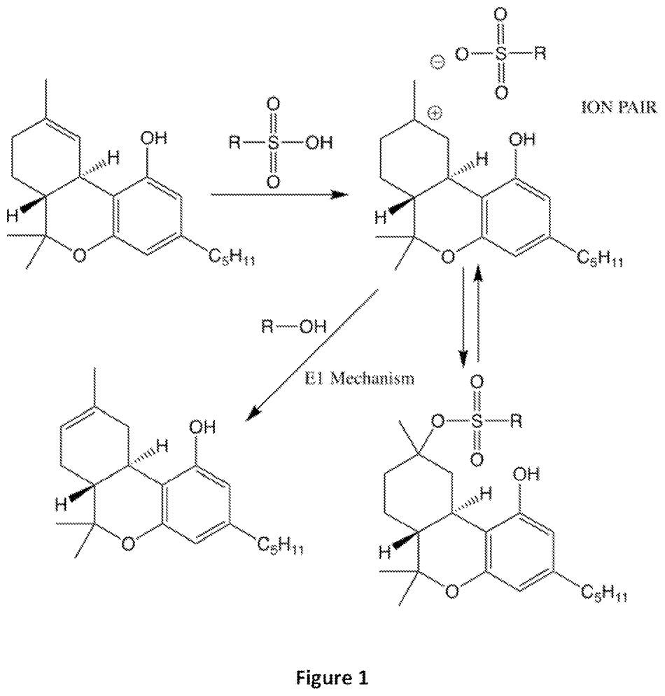

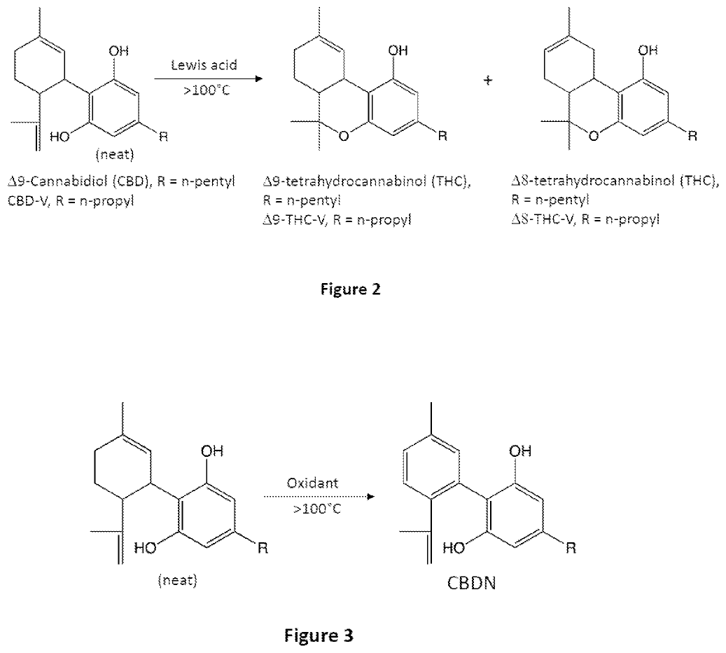

In addition to the above-described reactions with acid reagents (FIGS. 1-2), additional cannabinoids have been produced via reacting CBD with other reagents. With reference to FIGS. 3 and 4, it has been shown that reaction of CBD with an oxidant reagent only (i.e., in the absence of an acid) forms CBND, which reaction can optionally be performed in a solvent. Additionally, reaction of CBD with an oxidant reagent and acid reagent or an oxidant that also behaves as an acid forms CBN.

### Example 1

1 g (3.18 mmol) of pure CBD was place into a vial with 50 mg (0.52 mmol) of methane sulfonic acid (MSA). The vial was capped and then placed into an oven and then heated at 80° C. for 4 hours. After allowing the mixture to cool to around room temperature, the contents were analyzed by gas chromatography-mass spectrometry (GC-MS) and liquid chromatography-mass spectrometry (LC-MS). The results in Table 1 show minor products CBD-V, GBG and Δ-8 THC, and major products CBC and CBN, with higher concentration of CBC. No Δ-9 THC was detected.

### Example 2

The reaction conditions of Example 1 were repeated with 50 mg of dodecylbenzene sulfonic acid (DBSA) used as reagent in place of MSA. The results of Example 2 are also shown in Table 1 and indicate that the reaction failed to convert all of the CBD starting material.

### Example 3

The reaction conditions of Example 1 were repeated with 50 mg of toluene sulfonic acid (TSA) used as reagent in place of MSA. The results of Example 3 are shown in Table 1 and indicate that the major products of the reaction under these conditions were CBC, CBN and Δ-8 THC. Minor products were CBG and CBD-V. Interestingly, no Δ-9 product was detected.

### Example 4

The reaction conditions of Example 1 were repeated with 50 mg of polystyrene sulfonic acid (PSSA) used as reagent in place of MSA. The results of Example 4 are shown in Table 1, and show major products Δ-8 and Δ-9 THC with a preference for Δ-8 THC. Minor products were CBG and CBN. No CBD-V or CBC was detected.

### Example 10

10 g PSSA was added to a glass reaction tube purged with argon and under an argon flow in a heated sand bath held at 100° C., followed by addition of 50 g melted CBD at approximately 100° C. The reaction tube was maintained at 100° C. unstirred for 36 hours. The liquid contents were poured from the reaction tube into another glass tube yielding 45 g of crude reaction product. The crude reaction product was analyzed by liquid chromatography-mass spectrometry (LC-MS), which showed 6.24 mg/ml Δ-8 THC and 0.88 mg/ml Δ-9 THC. Thus, the stereoselectivity of Δ-8 THC:Δ-9 THC is approximately 71:1. Additionally, 5 g of reaction product remaining in the gummy PSSA after decantation was extracted with 40 ml hexane, followed by removal of hexanes via rotary evaporation. The product was analyzed by ¹H NMR and found to be at least 98% pure Δ-8 THC. No noticeable Δ-9 THC was detected in the NMR.

TABLE 1

Concentration (% of total)

| Product | Ex. 1 | Ex. 2 | Ex. 3 | Ex. 4 | Ex. 10 |
|---|---|---|---|---|---|
| CBD-V | 0.156 | 0.859 | 0.31 | — | — |
| CBG | 0.481 | — | 0.88 | 0.26 | — |
| CBN | 4.88 | — | 4.85 | 0.93 | — |
| Δ-8 THC | 1.02 | 2.81 | 12.6 | 66.5 | 99.2 |
| Δ-9 THC | — | 3.29 | — | 12.5 | 0.8 |
| CBC | 10.3 | 0.062 | 5.26 | — | — |
| CBD | — | 66.6 | — | — | — |

The results in Table 1 first show the efficacy of the disclosed solvent-free process for converting CBD to other cannabinoids simply in the presence of or upon addition of a Lewis Acid (in the examples, a sulfonic acid). The Examples additionally show that the relative yield of different cannabinoid products can vary significantly depending on the acid used to catalyze the neat reaction. Indeed, the solvent-free process allows direction of product yield simply by altering the acid that is used in catalytic amounts and eliminates difficulties associated with known synthetic processes that require solvent.

Notably, the reaction conditions in each of Examples are consistent with one another with exception to the identity of the respective acid reagent. In Examples 1-4, each acid reagent was present in at the same amount by weight (50 mg) and exposed to the same amount of CBD by weight (1 g) at the same temperature (80° C.) for the same duration (4 hours). The different acids produced starkly different compositions of the later cannabinoids (cannabinoids downstream from CBD in the typical biological conversion process), establishing that a specific Lewis Acid can produce a specific and varying result.

This is especially the case within Example 10, wherein concentration of acid (PSSA) relative to CBD, and increasing temperature and reaction duration relative to Example 4 drove conversion to 99.2% Δ-8 THC.

The four exemplary sulfonic acids above vary greatly in hydrophobicity and size. PSSA (the largest acid tested) was not soluble in liquid CBD up to 200° C.

As noted above, the inventive concepts of the method and instrument disclosed herein are not limited in terms of the form of the original CBD starting material. Embodiments of the solvent-free process and disclosed instruments allow conversion of CBD starting material in a variety of forms, including pure crystalline form, pure oil, gas, impure oil, in a plant including flax and hemp, on a solid support or inside a solid support.

Furthermore, with specific reference to FIG. 6, the inventive concepts of the method and instrument are not limited in terms of the exact starting material used. While the disclosure focuses on conversion of classic CBD starting material (i.e., wherein the R group is pentyl), a wide variety of alternate starting materials alternative R groups can be used, for example, wherein R is selected from the group consisting of methyl, ethyl, propyl, butyl, pentyl, hexyl, propyl-3-carboxylic acid, 1,1-dimethylheptyl, 4′-[2-(1H-1,2,3-triazol-yl)ethyl]-, 4′-(2-Morpholinoethyl)-, and 4′-(2-Ethoxyethyl)-. Compounds with these R groups are naturally occurring in hemp. Notably, none of the alternate R groups is affected by the reagents used within the disclosed reactions.

Additionally, shown in FIG. 6 are the different levels of oxidation products (first, second and third oxidation). As shown, as one oxidation, the major cannabinoid product is THC; on the second oxidation, the major product is CBND; and on the third oxidation, the major product is CBN. Δ-10 THC is formed under conditions that are generally opposite from those for forming Δ-8 THC. For example, a relatively small acid is reacted with temperature raised to drive formation of the thermodynamic product having the alkene in conjugation with the benzene ring.

The synthetic process is appropriate for use in bulk reactions, by melting CBD in the presence of an acid reagent or adding an acid reagent to melted CBD to yield later cannabinoids of varying concentrations. The process is suitable for two-phase reactions as well, such as by using PSSA coated on glass beads, silica or another inert support in the presence of CBD oil. In a two-phase reaction procedure, oils can be volatized by heat out of a plant, or off from a solid support, or from crystalline material. The volatized oil will pass through a solid-supported acid column such as PSSA coated on glass beads to convert CBD to Δ-8 THC, Δ-9 THC, CBN, and/or CBC. In such a process, reactions can be carried out on impure systems allowing for a boost in THC, CBN and/or CBC content from what was originally present in the oil or plant.

In the disclosed process described above, Lewis acids, such as the exemplary sulfonic acids, are used to convert CBD to Δ-9 THC via acid catalyzed etherification. With reference to FIG. 1, once Δ-9 THC is formed, the alkene can be protonated to generate a tertiary carbocation/sulfonate ion pair. In a polar medium, such as ethanol or water, the ion pair would simply dissociate. However, in the disclosed solvent-free process with only Δ-9 THC in CBD (no solvent or a two-phase system), the polarity is substantially lower, allowing a close-contact ion pair. The sulfonate forms a bond to the carbon with such close proximity that it yields an equilibrium between the carbocation and the sulfonate ester. Since each of CBC and Δ-9 THC is a large alcohol, they can be affected by the size of the R group as it approaches the carbocation. With larger R groups, β-elimination is strongly favored at the least sterically hindered adjacent carbon to the carbocation, thereby yielding Δ-8 THC (see Example 4). With smaller R groups, the reaction does not have as strong of a preference for the least sterically hindered hydrogen for abstraction to generate the alkene, thereby resulting in a larger yield of Δ-9 THC.

The below Examples 4-7 show conversion of CBD to other cannabinoids under different reaction conditions, including reactions utilizing clays, oxidants and combinations thereof.

### Example 5

1 g (3.18 mmol) CBD was placed in a 20 ml glass vessel and heated to 80° C. while stirring for 5 minutes. 2 equivalents SeO₂ was added to the CBD and the temperature increased to 130° C. for 72 hours. The results were analyzed via ¹H NMR spectroscopy and GC-MS. The results shown in Table 2 indicate that oxidant that also behaves as an acid converts CBD starting material to major product CBN and hydroxy DH-CBN-OH (an intermediate of CBN), and minor products CBND and intermediate compound(s).

### Example 6

1 g (3.18 mmol) CBD was placed in a 20 ml glass vessel and heated to 80° C. while stirring for 5 minutes. 2 equivalents SeO₂ was added to the CBD and the temperature increased to 200° C. for 10 minutes. The results were analyzed via ¹H NMR spectroscopy and GC-MS. The results shown in Table 2 indicate conversion of CBD to major products CBN and DH-CBN-OH, and minor product CBND and an intermediate, with an improved yield of CBN compared to Example 5.

### Example 7

1 g (3.18 mmol) CBD was placed in a 20 ml glass vessel and heated to 80° C. while stirring for 5 minutes. 3 equivalents SeO₂ and 0.5 g bentonite was added to the CBD and the temperature increased to 100° C. for 24 hours. The results were analyzed via ¹H NMR spectroscopy and GC-MS. The results shown in Table 2 show that oxidant and clay convert CBD starting material to major product DH-CBN-OH.

### Example 8

10 g (32 mmol) CBD was placed in a 20 ml glass vessel and heated to 80° C. while stirring for 5 minutes. 10 g bentonite was added to the CBD and the temperature increased to 130° C. for 24 hours. The results were analyzed via ¹H NMR spectroscopy and gas chromatography/mass spectrometry (GC-MS). The results shown in Table 2 show the efficacy of using clay that acts as a Lewis acid to convert CBD starting material to major products Δ-8 THC and Δ-9 THC with minor product CBN.

TABLE 2

Concentration (%)

| Product | Ex. 5 | Ex. 6 | Ex. 7 | Ex. 8 |
|---|---|---|---|---|
| CBD | 0 | 0 | 0 | 0 |
| CBND | 1.2 | 1 | 3 | 0 |
| CBN | 56 | 64 | 4 | 1.4 |
| Δ-8 THC | 0 | 0 | 0 | 56.4 |
| Δ-9 THC | 0 | 0 | 0 | 37.6 |
| DH-CBN-OH | 34 | 28 | 56 | 0 |
| Intermediate | 8.8 | 7 | 12 | 0 |
| Other | 0 | 0 | 25 | 4.6 |

The efficacy of clay reagents for use in converting CBD to other cannabinoids is notable as it allows substantial variations in conditions and product direction via modification of clays with other compounds, for example by adding an oxidant.

### Example 9

1 g (3.2 mmol) CBD was placed in a 20 ml glass vessel and heated to 80° C. while stirring for 5 minutes. 3 mmol FeCl₃ was added to the CBD and the temperature increased to 180° C. for 10 minutes. The results were analyzed via ¹H NMR spectroscopy, which results are shown in FIG. 5. As shown, the results indicate destruction of CBD starting material, polymerization and no presence of CBN or another cannabinoid product.

Notably, the respective reaction(s) described with reference to Examples 1-10 can also be performed in reactive distillation processes. In such a reaction, as is known in the art, a column is packed with a catalyst or catalyst is present within a distillation flask. Here, the CBD starting material reacts with the catalyst during the distillation process to form the respective product(s). Exemplary reactive distillation techniques are described in Kiss A., Jobson M., 2018, Taking reactive distillation to the next level of process intensification, Chemical Engineering Transactions, 69, 553-558 DOI: 10.3303/CET1869093.

Those skilled in the art will readily understand that the Examples do not serve to limit the invention, but rather offer illustrative evidence of the overall effectiveness of the disclosed process for converting CBD into cannabinoids and directing yields of different cannabinoids via altering a Lewis Acid reagent. The invention is not limited to the specific cannabinoids discussed herein, nor is it limited to the specific acids or even limited to sulfonic acids. For example, other appropriate Lewis acids include one or more from the group consisting of tin(IV) chloride, iron(III) bromide, iron(III) chloride, montmorillonite, bentonite, titanium(IV) chloride, titanium(IV) isopropoxide, boron trichloride, boron trichloride methyl sulfide, boron trifluoride, boron trifluoride dihydrate, boron trifluoride acetic acid, boron trifluoride acetonitrile, boron trifluoride tert-butyl methyl etherate, boron trifluoride dibutyl etherate, boron trifluoride diethyl etherate, aluminum chloride, aluminum isopropoxide, kaolinite, a zeolite, and zeolite-like metal-organic frameworks (ZMOFs).

Additionally, examples of clays that may be employed within the disclosed embodiments, either as a Lewis acid, oxidant or both, include kaolin-serpentine clays, such as kaolinite Al₂Si₂O₅(OH)₄, dickite and nacrite, polytypic varieties of kaolinite, halloysite (tubular, prismatic, rolled, pseudospherical, platy forms), chrysotile, antigorite, lizardite, greenalite; pyrophyllite-talc clays, including pyrophyllite (Al₂Si₄O₁₀(OH)₂), talc (Mg₃Si₄O₁₀(OH)₂), ferripyrophyllite; mica mineral clays, such as muscovite (KAl₂(Si₃Al)O₁₀(OH)₂), phlogopite KMg₃(Si₃Al)O₁₀(OH)₂ and biotite K(MgFe)₃(Si₃Al)O₁₀(OH)₂, illite, celadonite, glauconite; vermiculite; smectites, such as montmorillonite, beidellite, nontronite, volkonskoite, sauconite, stevensite, hectorite; chlorite (i.e., four end-member compositions, clinochlore, chamosite, pennantite, nimite), such as cookeite and donbassite; interstratified clay minerals, such as rectorite, tosudite, corrensite, aliettite, kulkeite; sepiolite; palygorskite; mogolite and allophane; pillared clays, including clays in which ions and/or molecules and/or polymers are intercalated into the clay between sheets, for example the exchange of ions originally present in a clay with other ions, absorption of non-water molecules into the clay structure other (i.e., hydrogen peroxide), intercalation of charged polymers that can either expand the clay structure or cause its exfoliation. Of particular interest to the disclosed embodiments, an oxidant (see below) may be mixed with a clay, yielding a pillared clay.

Similarly, other exemplary oxidants include chromates, such as ammonium dichromate, bis(tetrabutylammonium)dichromate, chromium (VI)oxide, potassium dichromate, pyridinium chlorochromate (PCC), pyridinium dichromate, sodium dichromate; iodine and hypervalent iodine compounds, such as bis(4-bromopheynl)iodonium triflate, bis(t-butylcarbonloxy)iodobenzene, bis(4-fluorophenyl)iodonium triflate, bis(4-methylphenyl)iodonium triflate, bis(pyridine)iodonium tetrafluoroborate, bis(trifluoroacetoxy)iodobenzene, bis(trifluoroacetoxy)iodobenzene purum, bis(trifluoroacetoxy)iodopentafluorobenzene, Dess-Martin periodinane, diacetoxyiodobenzene, diphenyliodonium chloride, hydroxy(tosyloxy)iodobenzene, 2-iodoxybenzoic acid, mIBX, (4-nitrophenyl)phenyliodonium triflate, periodic acid, penyl[3-(trifluoromethyl)phenyl]iondonium triflate, potassium 2-iodo-5-methylbenzenesulfonate, potassium periodate, sodium periodate, sodium (meta)periodate, sodium (para)periodate purum, tetrabutylammonium (meta)periodate; hypochlorites, such as calcium hypochlorite, sodium hypochlorite; osmium compounds, such as osmium tetroxide; perchlorates, such as aluminum perchlorate nonahydrate, barium perchlorate, cadmium perchlorate hydrate, calcium perchlorate tetrahydrate, cesium perchlorate, copper (II) perchlorate, (dansylaminoethyl)trimethylammonium perchlorate, N-hydroxytetrachlorophthalmide, Indium (III) perchlorate hydrate, Iron (II) perchlorate hydrate, Iron(III) perchlorate hydrate, lead (II) perchlorate hydrate, lead (II) perchlorate trihydrate, lithium perchlorate, magnesium perchlorate, magnesium perchlorate hexahydrate, magnesium perchlorate hydrate, mercury (II) perchlorate hydrate, nickel (II) perchlorate hexahydrate, perchloric acid, silver perchlorate, silver perchlorate hydrate, sodium perchlorate, sodium perchlorate hydrate, tetrabutylammonium perchlorate, zinc perchlorate hexahydrate; peroxides, such as benzoyl peroxide, 2-butanone peroxide, t-butylhydroperoxide, calcium peroxide, cumene hydroperoxide, dicumyl peroxide, hydrogen peroxide in water (15 to 30% hydrogen peroxide), hydrogen peroxide urea adduct, lithium peroxide, Lauroyl peroxide, magnesium peroxide, nickel peroxide, nickel (II) peroxide hydrate, sodium peroxide, strontium peroxide, urea hydrogen peroxide, zinc peroxide; peroxyacids and salts, such as 3-chloroperbenzoic acid (mcpba), magnesium bis(monoperoxyphthalate)hexahydrate, magnesium monoperoxyphthalate hexahydrate, peracetic acid (10 to 40% in dilute acetic acid); selenium and sulfur containing compounds, such as sulfur, selenium, ammonium persulfate, potassium nitrosodisulfonate, potassium peroxodisulfate, potassium persulfate, sodium persulfate, sulfur trioxide N, N-dimethylformamide complex, sulfur trioxide pyridine complex, sulfur trioxide triethylamine complex, sulfur trioxide trimethyl amine complex, TBSAB; and other known oxidizing agents, such as ammonium cerium (IV) nitrates, ammonium phosphomolybdate hydrate, 2-azaadamantane-N-oxyl, bis(triphenylsilyl) chromate, N-t-butylbenzenesulfenamide, chloramine T trihydrates, chloranil purum, N-chlorobenzene fsulfonamide sodium salt, 2,3-dichloro-5,6-dicyano-p-benzoquinone, N,N-dichloro-p-sulfonamide, ethyl chlrooxoacetate, 8-ethylquinoline N-oxide, N-hydroxytetrachlorophthalimide, KetoABNO, methyl chlorooxoacetate, 4-methylmorpholine N-oxide, nitrosyl tetrafluoroborate, oxalyl bromide, oxalyl chloride, phosphomolybdic acid, potassium perruthenate, selenium dioxide, sodium dichloroisocyanurate, sodium percarbonate avail, sodium permanganate, sodium phosphomolybdate hydrate, TEMPO, tetracyanoethylene, 2,2,6,6-tetramethyl-4-[1-oxo-6(triethylammonio)hexylamino]-1-piperidinyloxy bromide, tetrapropylammonium perruthenate, trimethylamine N-oxide, trimethylamine N-oxide dihydrate; and oxygen. Notably, persulfates have shown to be particularly effective reagents for converting CBD to CBN.

Exemplary solvents for optional use within the process of converting CBD to CBND in the presence of oxidant only include polar aprotic solvents, including N,N-dimethylformamide, N,N-dimethylacetamide and dimethylsulfoxide; ethers, including tetrahydrofuran, dimethoxyethane, 1,4-dioxane and diethyl ether; and nonpolar solvents, including methylene chloride, chloroform, hexane, heptane and o-dichlorobenzene.

While a preferred embodiment has been set forth for purposes of illustration, the foregoing description should not be deemed a limitation of the invention herein. Accordingly, various modifications, adaptations and alternatives may occur to one skilled in the art without departing from the spirit of the invention and scope of the claimed coverage.

1. A method for converting a cannabidiol-like (CBD-like) starting material to one or more cannabinoids, comprising:
providing a CBD-like starting material; and
exposing the CBD-like starting material to an acid reagent in the absence of solvent, or
exposing the CBD-like starting material to an oxidant reagent in the absence of solvent, or
exposing the CBD-like starting material to an oxidant reagent and an acid reagent in the absence of solvent, or
exposing the CBD-like starting material to an oxidant reagent that also behaves as an acid in the absence of solvent,
to yield one or more cannabinoid products, wherein
the CBD-like starting material is: 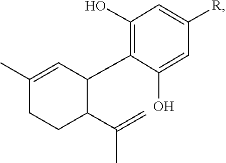
wherein
R is selected from the group consisting of methyl, ethyl, propyl, butyl, pentyl, hexyl, propyl-3-carboxylic acid, 1,1-dimethylheptyl, 4′-[2-(1H-1,2,3-triazol-yl)ethyl]-, 4′-(2-Morpholinoethyl)- and 4′-(2-Ethoxyethyl)-.

2. The method of claim 1, wherein the CBD-like starting material is exposed to an acid reagent present in a catalytic amount.

3. The method of claim 1, wherein the CBD-like starting material is exposed to the acid reagent, oxidant reagent or both in a reactive distillation process.

4. The method of claim 1, wherein the CBD-like starting material is substantially pure.

5. The method of claim 1, wherein the CBD-like starting material is part of an impure material containing other cannabis-derived compounds.

6. The method of claim 1, wherein the acid reagent or oxidant reagent or both dissolves in the CBD-like starting material.

7. The method of claim 1, wherein the acid reagent or oxidant reagent or both is insoluble in the CBD-like starting material.

8. The method of claim 1, wherein the CBD-like starting material is CBD and one or more cannabinoid products is selected from the group consisting of Δ-8 THC, Δ-9 THC, Δ-10 THC, CBC and CBN.

9. The method claim 1, wherein the acid reagent in water has a pH no greater than 3.

10. The method of claim 1, wherein the CBD-like starting material is exposed to an acid reagent selected from one or more from the group consisting of sulfonic acids, tin(IV) chloride, iron(III) bromide, iron(III) chloride, montmorillonite, bentonite, titanium(IV) chloride, titanium(IV) isopropoxide, boron trichloride, boron trichloride methyl sulfide, boron trifluoride, boron trifluoride dihydrate, boron trifluoride acetic acid, boron trifluoride acetonitrile, boron trifluoride tert-butyl methyl etherate, boron trifluoride dibutyl etherate, boron trifluoride diethyl etherate, aluminum chloride, aluminum isopropoxide, kaolinite, a zeolite, and zeolite-like metal-organic frameworks (ZMOFs).

11. The method of claim 1, wherein the CBD-like starting material is exposed to an oxidant reagent selected from one or more from the group consisting of a chromate, including ammonium dichromate, bis(tetrabutylammonium)dichromate, chromium (VI)oxide, potassium dichromate, pyridinium chlorochromate (PCC), pyridinium dichromate, sodium dichromate; iodine and hypervalent iodine compounds, including bis(4-bromopheynl)iodonium triflate, bis(t-butylcarbonloxy)iodobenzene, bis(4-fluorophenyl)iodonium triflate, bis(4-methylphenyl)iodonium triflate, bis(pyridine)iodonium tetrafluoroborate, bis(trifluoroacetoxy)iodobenzene, bis(trifluoroacetoxy)iodobenzene purum, bis(trifluoroacetoxy)iodopentafluorobenzene, Dess-Martin periodinane, diacetoxyiodobenzene, diphenyliodonium chloride, hydroxy(tosyloxy)iodobenzene, 2-iodoxybenzoic acid, mIBX, (4-nitrophenyl)phenyliodonium triflate, periodic acid, penyl[3-(trifluoromethyl)phenyl]iondonium triflate, potassium 2-iodo-5-methylbenzenesulfonate, potassium periodate, sodium periodate, sodium (meta)periodate, sodium (para)periodate purum, tetrabutylammonium (meta)periodate; hypochlorites, including calcium hypochlorite, sodium hypochlorite; osmium compounds, including osmium tetroxide; perchlorates, including aluminum perchlorate nonahydrate, barium perchlorate, cadmium perchlorate hydrate, calcium perchlorate tetrahydrate, cesium perchlorate, copper (II) perchlorate, (dansylaminoethyl)trimethylammonium perchlorate, N-hydroxytetrachlorophthalmide, Indium (III) perchlorate hydrate, Iron (II) perchlorate hydrate, Iron(III) perchlorate hydrate, lead (II) perchlorate hydrate, lead (II) perchlorate trihydrate, lithium perchlorate, magnesium perchlorate, magnesium perchlorate hexahydrate, magnesium perchlorate hydrate, mercury (II) perchlorate hydrate, nickel (II) perchlorate hexahydrate, perchloric acid, silver perchlorate, silver perchlorate hydrate, sodium perchlorate, sodium perchlorate hydrate, tetrabutylammonium perchlorate, zinc perchlorate hexahydrate; peroxides, including benzoyl peroxide, 2-butanone peroxide, t-butylhydroperoxide, calcium peroxide, cumene hydroperoxide, dicumyl peroxide, hydrogen peroxide in water (15 to 30% hydrogen peroxide), hydrogen peroxide urea adduct, lithium peroxide, Lauroyl peroxide, magnesium peroxide, nickel peroxide, nickel (II) peroxide hydrate, sodium peroxide, strontium peroxide, urea hydrogen peroxide, zinc peroxide; peroxyacids and salts, including 3-chloroperbenzoic acid (mcpba), magnesium bis(monoperoxyphthalate)hexahydrate, magnesium monoperoxyphthalate hexahydrate, peracetic acid (10 to 40% in dilute acetic acid); selenium containing compounds, selenium, ammonium cerium (IV) nitrates, ammonium phosphomolybdate hydrate, 2-azaadamantane-N-oxyl, bis(triphenylsilyl) chromate, chloramine T trihydrates, chloranil purum, 2,3-dichloro-5,6-dicyano-p-benzoquinone, ethyl chlrooxoacetate, 8-ethylquinoline N-oxide, N-hydroxytetrachlorophthalimide, KetoABNO, methyl chlorooxoacetate, 4-methylmorpholine N-oxide, nitrosyl tetrafluoroborate, oxalyl bromide, oxalyl chloride, phosphomolybdic acid, potassium perruthenate, selenium dioxide, sodium dichloroisocyanurate, sodium percarbonate avail, sodium permanganate,,sodium phosphomolybdate hydrate, TEMPO, tetracyanoethylene, 2,2,6,6-tetramethyl-4-[1-oxo-6(triethylammonio)hexylamino]-1-piperidinyloxy bromide, tetrapropylammonium perruthenate, trimethylamine N-oxide, trimethylamine N-oxide dihydrate; and oxygen.

12. The method of claim 1, wherein the CBD-like starting material is exposed to a clay.

13. The method of claim 12, wherein the clay is selected from one or more from the group consisting of kaolin-serpentine clays, including kaolinite Al₂Si₂O₅(OH)₄, dickite and nacrite, polytypic varieties of kaolinite, halloysite (tubular, prismatic, rolled, pseudospherical, platy forms), chrysotile, antigorite, lizardite, greenalite; pyrophyllite-talc clays, including pyrophyllite (Al₂Si₄O₁₀(OH)₂), talc (Mg₃Si₄O₁₀(OH)₂), ferripyrophyllite; mica mineral clays, including muscovite (KAl₂(Si₃Al)O₁₀(OH)₂), phlogopite KMg₃(Si₃Al)O₁₀(OH)₂ and biotite K(MgFe)₃(Si₃Al)O₁₀(OH)₂, illite, celadonite, glauconite; vermiculite; smectites, including montmorillonite, beidellite, nontronite, volkonskoite, sauconite, stevensite, hectorite; chlorite (i.e., four end-member compositions, clinochlore, chamosite, pennantite, nimite), including cookeite and donbassite; interstratified clay minerals, including rectorite, tosudite, corrensite, aliettite, kulkeite; sepiolite; palygorskite; mogolite and allophane; and pillared clays.

14. The method of claim 10, wherein the acid reagent is one or more from the group consisting of hydrochloric acid, hydrobromic acid, hydroiodic acid, sulfuric acid, nitric acid, phosphoric acid, polystyrenesulfonic acid, polyphosphonic acid, an alkanesulfonic acid and a sulfonation product of an aromatic ring.

15. The method of claim 1, wherein
the CBD-like starting material starting material is CBD,
the CBD is exposed to PSSA at elevated temperature, and
the major cannabinoid product is Δ-8 THC.

16. The method of claim 15, wherein the elevated temperature is within a range of 70° C.-140° C.

17. The method of claim 15, wherein the Δ-8 THC cannabinoid product is present in an amount relative to Δ-9 THC of between 50:1 and 1000:1.

18. The method of claim 1, wherein
the CBD-like starting material is CBD,
the CBD is exposed to TSA at elevated temperature, and
the major cannabinoid products are CBN, Δ-8 THC and CBC.

19. The method of claim 1, wherein
the CBD-like starting material is CBD,
the CBD is exposed to DBSA at elevated temperature, and
the major cannabinoid products are CBN and CBC.

20. The method of claim 1, wherein the CBD-like starting material is exposed to a sterically hindered acid reagent to yield Δ-8 THC and Δ-9 THC in a relative amount Δ-8 THC:Δ-9 THC of 5:1 or greater.

21-37. (canceled)
